(19) United States
(12) Patent Application Publication
(10) Pub. No.: US 2016/0083898 A1
(43) Pub. Date: Mar. 24, 2016

(54) PROCESS AND SYSTEM FOR LASER-CUTTING A SHAPE IN A MOVING WEB

(71) Applicant: LASERAX INC., Québec (CA)

(72) Inventors: Xavier Pruneau Godmaire, Saint-Lambert-de-Lauzon (CA); Alex Fraser, Quebec (CA)

(21) Appl. No.: 14/891,152

(22) PCT Filed: Apr. 30, 2014
(86) PCT No.: PCT/CA2014/050411

§ 371 Date: Nov. 13, 2015

Related U.S. Application Data
(60) Provisional application No. 61/824,161, filed on May 16, 2013.

(51) Int. Cl. D06H 7/22; A61F 13/15; B23K 26/08; B23K 26/70; B23K 26/38; B23K 26/402

(52) U.S. Cl. CPC D06H 7/221; B23K 26/38; B23K 26/402; B23K 26/083; B23K 26/0869; B23K 26/702; A61F 13/15723

(57) ABSTRACT

The process can include moving the web across a laser-cutting window in a longitudinal direction while maintaining a main tension in the web, the main tension being in the longitudinal direction; applying a specific tension to at least a portion of the web; and cutting a portion of the shape by moving the laser beam along the portion of the shape, within the laser-cutting window, during said moving of the web, and while maintaining at least a portion of the specific tension at the laser beam in an orientation different than the instantaneous orientation of movement of the laser beam.

### CROSS-REFERENCE TO RELATED APPLICATIONS

This application is a non-provisional of U.S. Patent Provisional Application No. 61/824,161, filed on May 16, 2013, the contents of which are incorporated by reference.

### FIELD

The improvements generally relate to the field of cutting shapes in material provided in the form of a moving web, and more specifically relate to a method of cutting using a laser.

### BACKGROUND

Cutting regular shapes in material provided in the form of a web was typically done using blades. This was satisfactory to a certain degree yet involved certain constraints. For instance, blades required regular maintenance and changing the cut shape typically required changing the blade set altogether. There thus remained room for improvement, particularly in terms of addressing the versatility and/or maintenance costs associated with such systems.

### SUMMARY

In accordance with one aspect, there is provided a process of laser-cutting a shape in a moving web, the process comprising: moving the web across a laser-cutting window in a longitudinal direction while maintaining a main tension in the web, the main tension being in the longitudinal direction; applying a specific tension to at least a portion of the web; and cutting a portion of the shape by moving the laser beam along the portion of the shape, within the laser-cutting window, during said moving of the web, and while maintaining at least a portion of the specific tension at the laser beam in an orientation different than the instantaneous orientation of movement of the laser beam.

The portion of the shape can be a subsequent portion of the shape, and the process can further include, prior to the step of cutting a subsequent portion of the shape, cutting a former portion of the shape by moving the laser beam along the former portion of the shape, within the laser-cutting window, during said moving of the web, and thereby freeing a portion of the web from the main tension, and the step of applying a specific tension can include applying a specific tension to said freed portion of the web.

The subsequent portion of the shape can be at least partially longitudinally aligned with the former portion of the shape.

The step of applying a specific tension can include applying the specific tension in an orientation different than the longitudinal orientation, and wherein the step of cutting a portion of the shape includes moving the laser beam in the longitudinal orientation.

The specific tension can be applied in a direction having an acute angle relative to the longitudinal direction.

The step of applying a specific tension to the freed portion of the web can include engaging the freed portion of the web between nip rollers.

In accordance with another aspect, there is provided a system of laser-cutting a shape in a moving web, the system comprising: means for moving the web across a laser-cutting window in a longitudinal direction while maintaining a main tension in the web, the main tension being in the longitudinal direction; means for applying a specific tension to at least a portion of the web; and means for cutting a portion of the shape by moving the laser beam along the portion of the shape, within the laser-cutting window, during said moving of the web, and while maintaining at least a portion of the specific tension at the laser beam in an orientation different than the instantaneous orientation of movement of the laser beam.

Many further features and combinations thereof concerning the present improvements will appear to those skilled in the art following a reading of the instant disclosure.

### DESCRIPTION OF THE FIGURES

In the figures,

FIG. 1 is a top plan view of an example of a shape to be cut in material provided in the form of a web;

FIGS. 2A to 2C are sequential views showing a web moving across a laser-cutting window;

FIG. 3 is a top plan view showing applying a specific tension to a freed portion of the web using nip rollers;

FIGS. 4A to 4C are sequential views showing laser-cutting of another shape in a web;

FIGS. 5A and 5B show variants of application of specific tension to the web;

FIG. 6 shows an example of a shape to be cut using two laser beams; and

FIG. 7 shows an other example of a shape to be cut using two laser beams.

### DETAILED DESCRIPTION

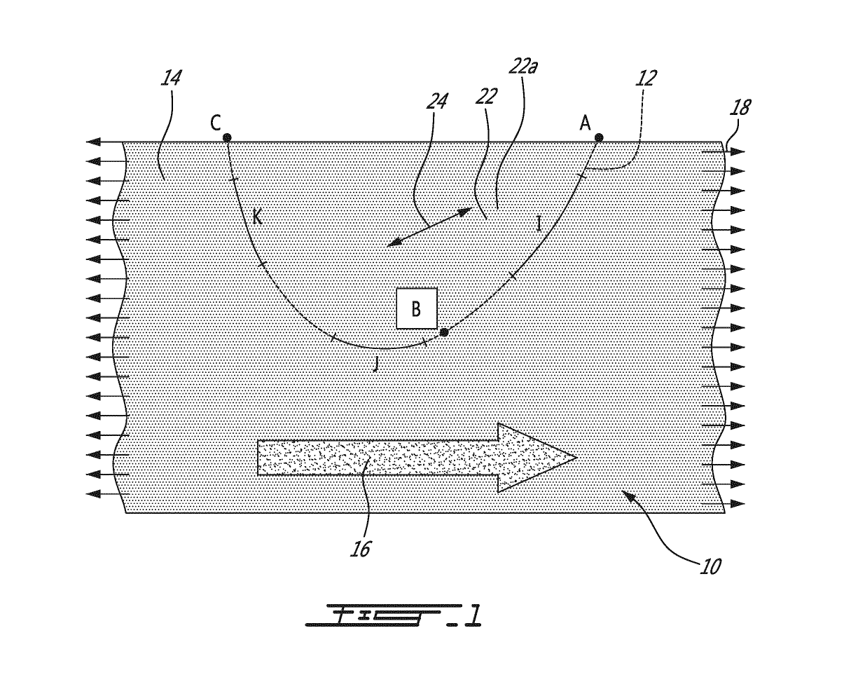

FIG. 1 shows a first example of a shape 12 to be cut out of material 14 provided in the form of a moving web 10, where the web 10 is moved in a direction referred to herein as the longitudinal direction 16. This shape 12 can be cut, for instance, by cutting from point A to point B and then to point C. Cutting from point C to point A, can also be possible in some embodiments but was not preferred in this example since it imparted more requirements on the system. To be satisfactory in terms of laser power requirement and/or cut quality, it was found that cutting with a laser should be done while a tension was maintained in the material, with at least a component of the tension being transversal to the instantaneous cutting orientation of the laser beam at all times.

Materials provided in the form of a web 10 typically have a main tension 18 provided naturally in the web 10 and being oriented in the longitudinal orientation 16. This main tension is typically imparted to the web 10 by the web movement system, typically including a plurality of rollers.

Henceforth, cutting a first portion of a shape such as shown in FIG. 1, e.g. a portion found between points A and B for instance, can be done effectively using a laser without any particular adaptations. An example process can be as follows:

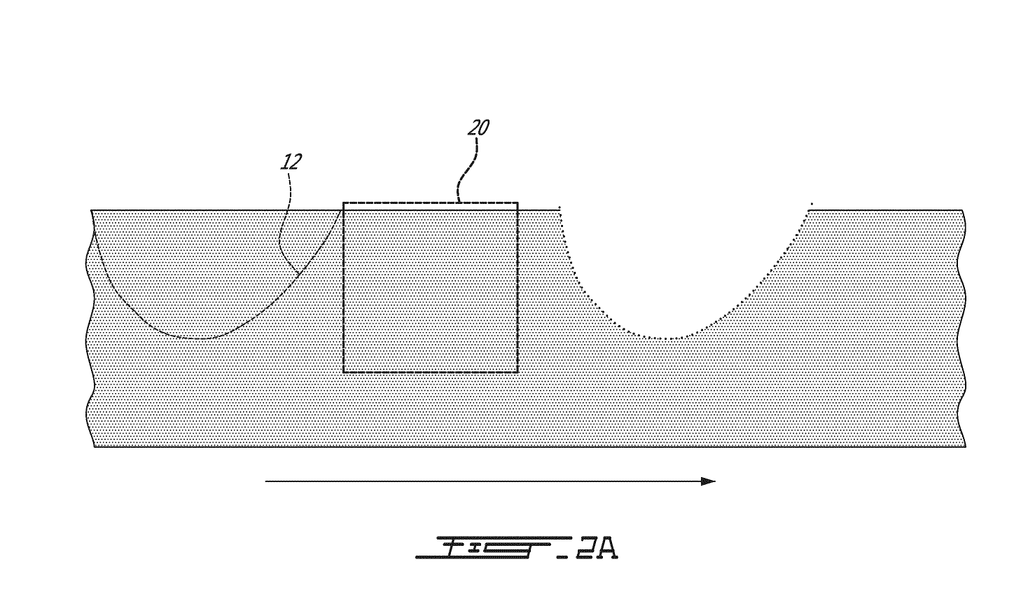

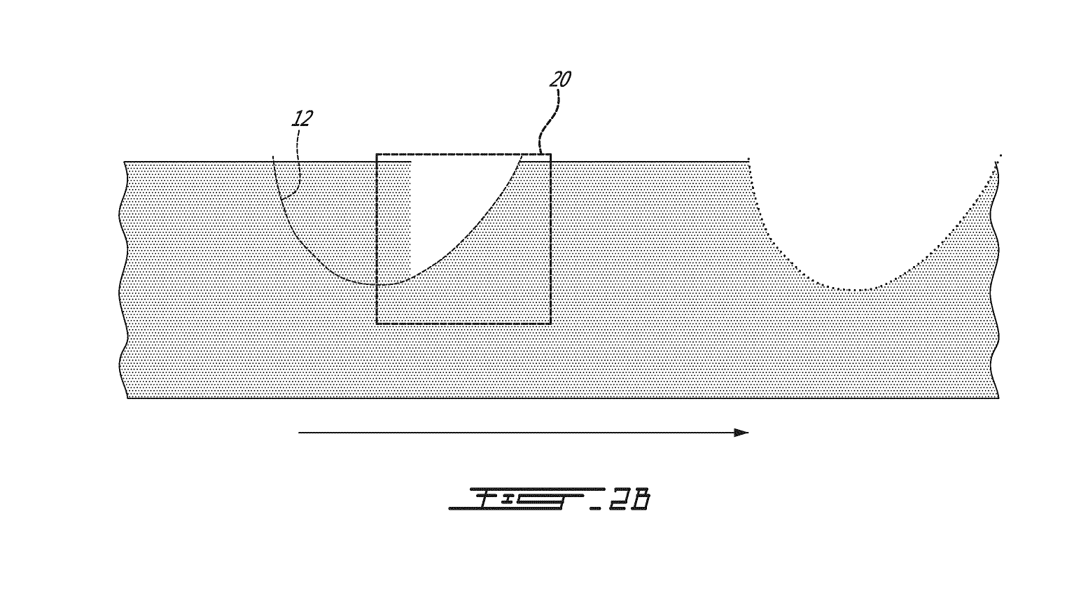

The web 10 is moved across a laser-cutting window 20 which can be fixed relative to the ground and which can represent the limits within which a laser beam of the laser can be moved, for instance. Henceforth, a shape 12 to be cut progressively travels across the laser-cutting window 20, such as shown in FIGS. 2A to 2C, during which the laser beam is moved along the shape 12, within the laser-cutting window 20, which typically also involves moving the laser beam relative to the laser-cutting window 20 (in one or two dimensions). In practice, it will be understood that since the power requirement of the laser depends on the speed of instantaneous movement of the laser beam along the portion of the shape 12, it can be preferred to maintain a constant speed of the laser beam along the shape. This can be achieved by software which can be used to control the movement of the laser beam at a variable speed along a laser beam path relative the laser-cutting window 20, which will correspond to moving the laser beam at a constant speed along the shape when taking the speed of movement of the web into consideration. In this specification, the expression laser beam is used generally as referring to a spot which is being aimed by the laser system, independently of whether the laser beam is continuous or pulsed. The speed of the laser beam relative to the web is often referred to in the art as the ‘marking speed’.

Referring back to FIG. 1, it will be understood that while cutting a first portion, e.g. portion I, the main tension 18 continuously applies a tension in the web 10 at the laser beam as the laser beam travels along the portion I, and the main tension 18 being in the longitudinal orientation 16, remains in an orientation which is substantially different (i.e. non-parallel) than the instantaneous orientation of movement of the laser beam along the entire portion I. This leads to satisfactory laser-cutting ability along the portion I.

At one point along the shape ABC however, which can be after reaching point B in this example, the instantaneous orientation of movement of the laser beam becomes sufficiently parallel to the longitudinal orientation 16 of the main tension 18 to lead to unsatisfactory laser-cutting.

Moreover, cutting the portion AB frees a portion of the web immediately ‘upstream’ of the cut from the main tension 18 in the web 10. This can lead to creating a portion 22 of the web 10 where the tension is insufficient to provide satisfactory cutting, and this portion 22 of the web 10 can extend to a certain distance longitudinally upstream of the cut. Such a portion 22 of the web 10 which is substantially freed from the main tension 18 in the web 10 will be referred to herein as a freed portion 22a of the web 10.

It was found that a subsequent portion of the shape such as portion J or portion K along the path BC for instance, can nonetheless be laser-cut in a satisfactory manner. This can be achieved by applying a specific tension 24 to the freed portion 22a of the web prior to moving the laser beam along such portions. The specific tension 24 can allow maintaining a satisfactory tension at the laser beam in an orientation different than the instantaneous orientation of movement of the laser beam even when the laser beam is moved in the longitudinal orientation 16 such as along portion J, or along a portion such as K when the main tension 18 is lost by former cutting of the web 10 upstream or downstream of the portion (K) within a certain distance, for instance.

Cutting longitudinally, e.g. along portion J, can be achieved satisfactorily by applying a specific tension to the web 10 in the transversal orientation, whereas cutting a portion of the web 10 which is longitudinally aligned with a previously cut portion, and where the main tension 18 has been lost, can be achieved satisfactorily by applying a specific tension to the freed portion 22a of the web 10.

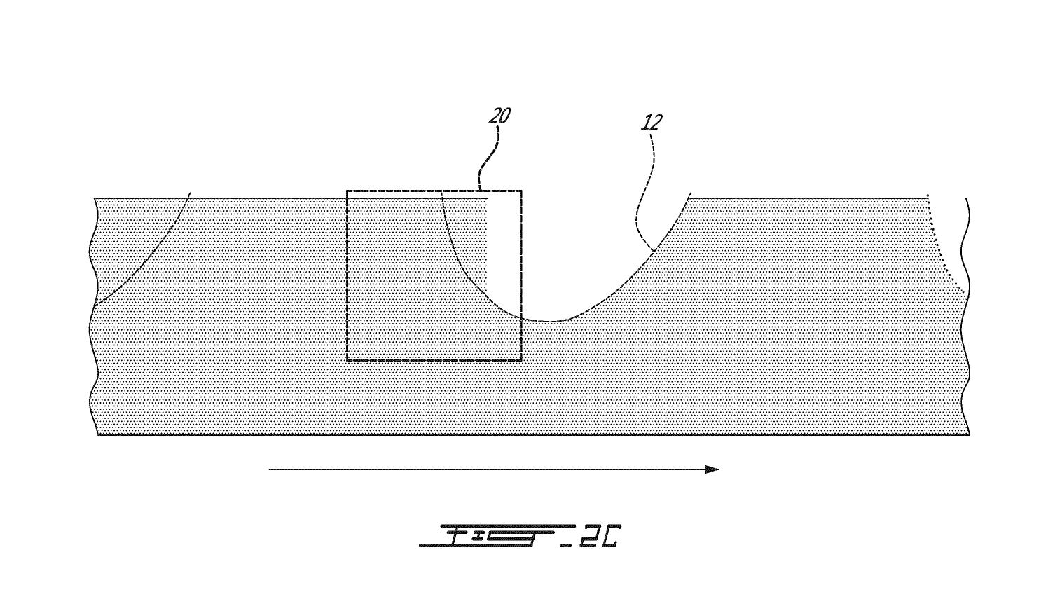

In one example embodiment illustrated at FIG. 3, a specific tension 24a is applied to the freed portion 22a in a direction having an acute angle θ relative to the longitudinal direction. By adequately selecting the acute angle θ as a function of the shape to be cut, the single specific tension 24a allowed to cut both portions J and K in this case. In this embodiment, the specific tension 24a is applied to the web 10 using nip rollers 26 on both sides of the web 10 which ‘catch’ the freed portion 22a of the web 10 prior cutting of the segment BC. The nip rolls can be an assembly including two rolls rotating in opposite directions, on opposite sides of the freed portion 22a. The rolls can be adjusted in close contact and can be engineered with a particular surface finish in order to increase the grip on the trims. In order to apply a satisfactory amount of specific tension, the tangential speed of the nip rolls can be adjusted to be slightly higher than the longitudinal speed of the moving web—an elongation of the material in the order of 4% can be satisfactory, for instance. In this embodiment, the nip rolls are placed at a small angle relative to the direction perpendicular to the longitudinal direction which allows generating a tension in the trim with a component perpendicular to the cutting direction. In alternate embodiment, such a specific tension can be applied using an other mechanism than a nip roller, such as vacuum suction for instance, and also have the effect of pulling the freed portion of the web from the remaining portion of the web. Still alternately, the specific tension can result from a combination of mechanisms, applying tension in different directions, for instance, such as a nip roller applying a tension in the longitudinal orientation and a banana roll or banana bar applying a tension in the transversal orientation to name an additional example. It will be understood that when a component of tension is applied in one transversal direction, e.g. by a nip roller adjacent a first edge of the web and oriented obliquely relative to the main tension, it can be preferred to compensate this tension with a mirrored tension applied in the opposite transversal direction, e.g. by another nip roller located adjacent a second edge of the web and oriented in the opposite oblique orientation relative to the main tension, to avoid ‘pulling’ the web to one side or another. Such a mirrored tension can be readily applied simply by repeating the same ‘catching’ and ‘pulling’ of the freed portion on a corresponding second shape on the other side of the web, such as shape 54, shown in FIG. 7, for instance.

In the example illustrated in FIG. 3, we can consider point B as being the cutting position where the main tension becomes too small to ensure effective cutting along path J and X as the transverse distance between this cutting position and the edge of the web in the orientation perpendicular to the material movement. We can also consider Y as being the longitudinal distance between a point of entry on the web and the location of application of the specific tension 24a, the web speed as being Z, and the laser beam transverse mean speed from A to B, as being Z′. In such a case, it can be preferred that the controller of the laser beam ensure that:

X/Z′>Y/Z+t (1)

Where t is the amount of time required to build a satisfactory amount of specific tension.

Moreover, in order for the cutting process to be realized with a constant relative speed between the laser beam and the web and in order to achieve the illustrated cut shape, the laser beam motion system should allow the laser beam to undertake a path that is not limited to the transversal orientation. An example two-dimensional beam path 30 is shown for illustrative purposes, in which case relation (1) is also preferably preserved.

It will be noted that in an embodiment such as shown in FIG. 3, the nip rollers will likely be configured in alignment with the plane of the web. However, in alternate embodiments, it will be understood that the nip rollers can be positioned above or below the plane of the web, for instance, and apply the specific tension with an additional angle oriented obliquely relative to the plane of the web.

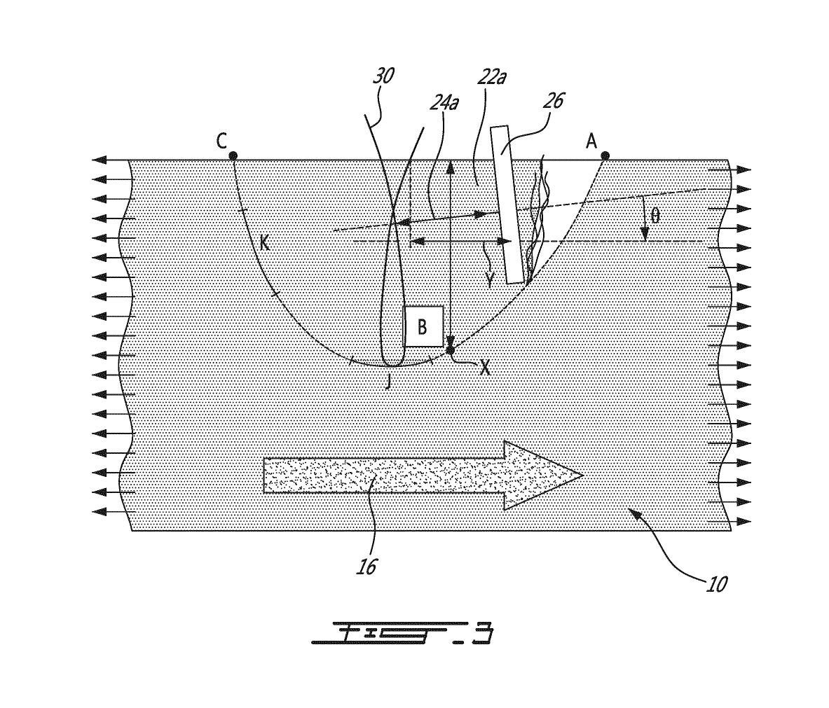

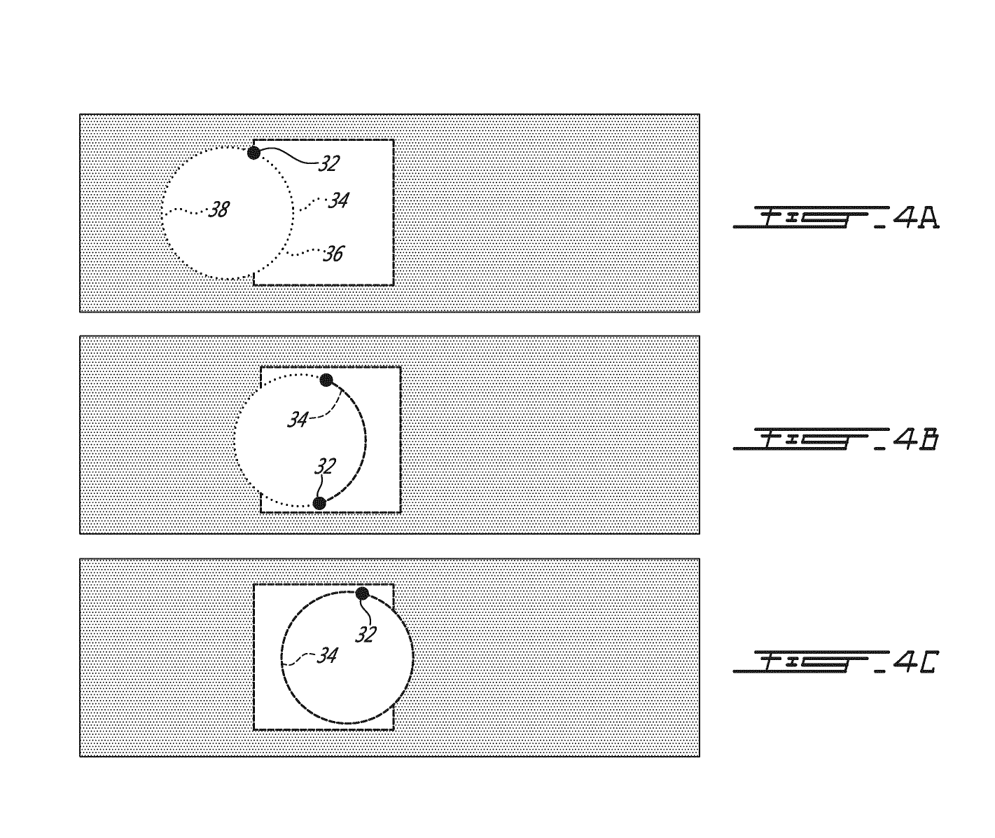

FIGS. 4A to 4C illustrate another example embodiment where a shape is laser-cut in a moving web of material under a main longitudinal tension. As can be seen by the sequence illustrated, a single laser beam 32 is moved along a closed shape 34 to fully cut the desired shape from the web in this embodiment. In alternate embodiments, a combination of laser beams, such as two laser beams following two distinct portions of the shape, can be used instead of a single laser beam.

In the embodiment shown in FIGS. 4A to 4C, it can be understood that the main tension in the web can be sufficient in itself to laser-cut the downstream portion 36 illustrated as being cut from FIG. 4A to FIG. 4B. However, the upstream portion 38 being cut between FIGS. 4B and 4C includes portions which are oriented longitudinally and a portion for which the main tension was lost due to the cutting of the downstream portion 36.

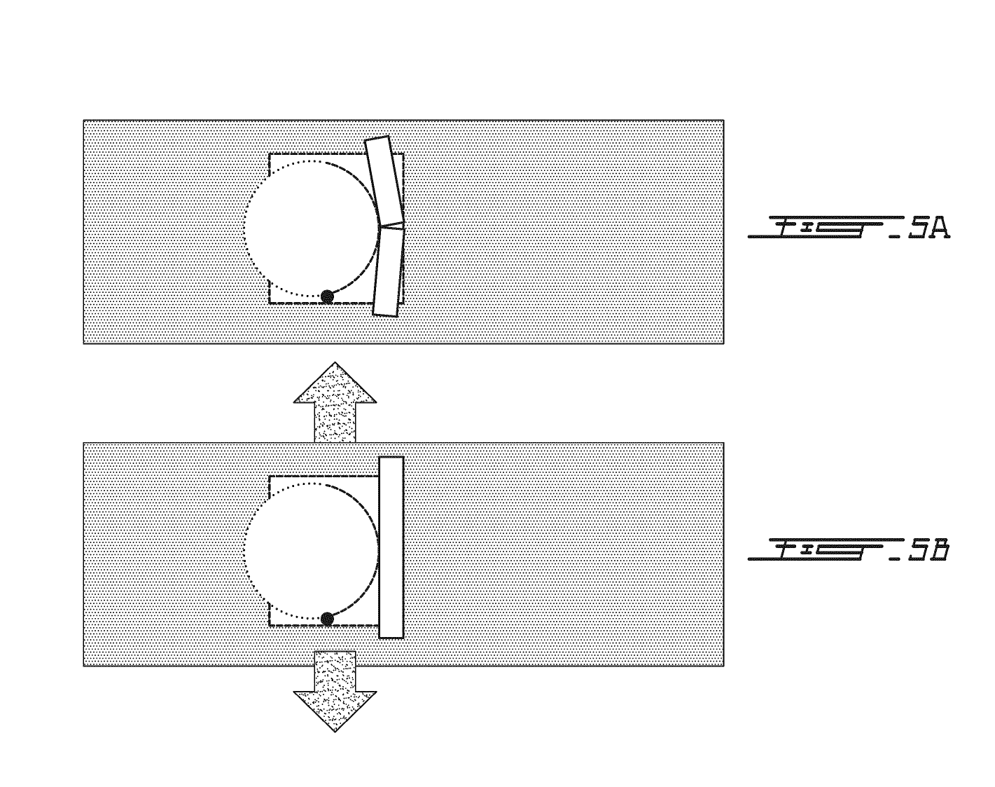

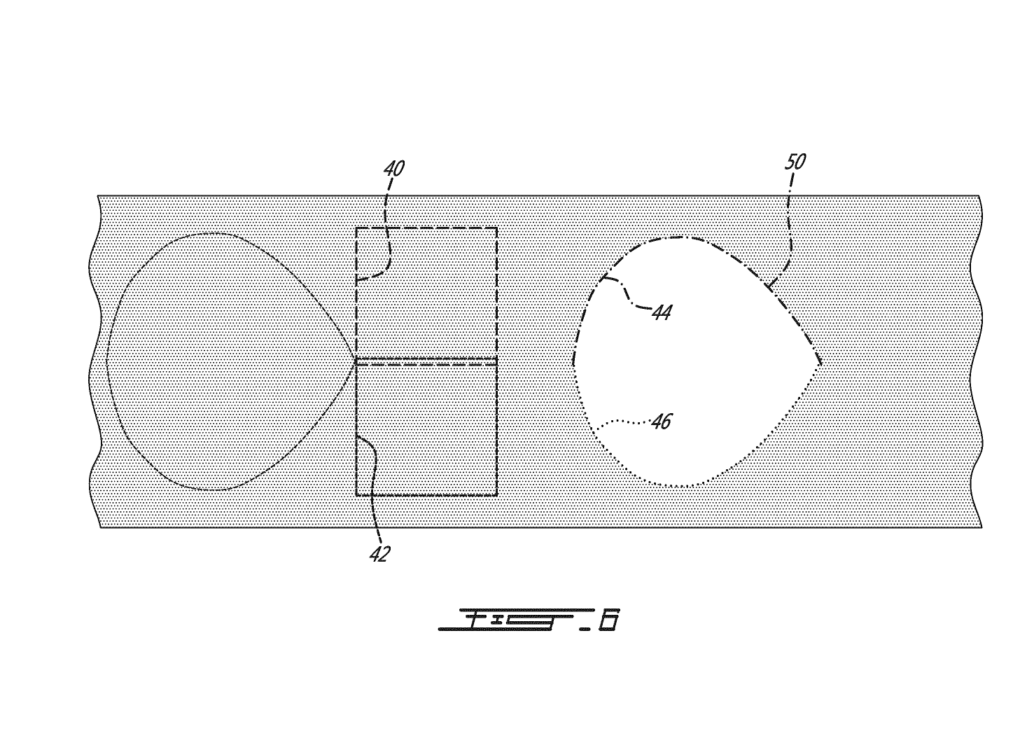

In this specific embodiment, satisfactory cutting of the upstream portion can be achieved by a combination of two sets of nip rollers each inclined by an acute angle relative to the longitudinal orientation, such as shown in FIG. 5A, or by a combination of a transversally-oriented set of nip rollers applying a longitudinal tension to the freed portion and a separate mechanism which applies transversal tension, such as shown in FIG. 5B, to name two examples.

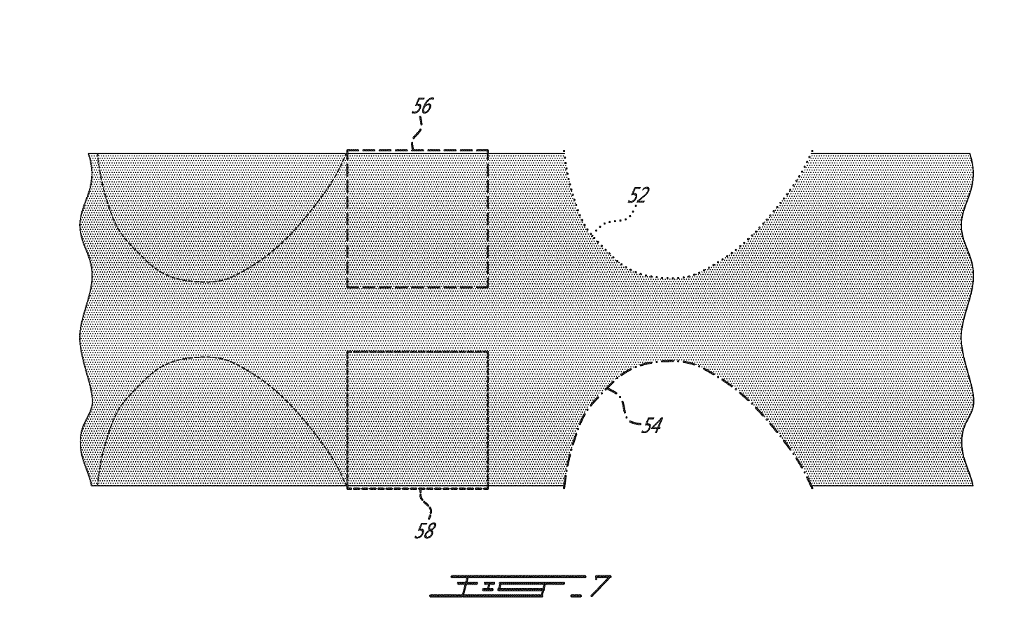

FIG. 6 illustrates another example of a shape to be cut in a web, in which case two different laser beams are used, each associated to a corresponding laser-cutting window and a corresponding transversal side 44, 46 of the shape 50.

FIG. 7 illustrates another example of a web in which a repeated pattern of two shapes 52, 54 are to be cut on corresponding edges of the web, each associated to a corresponding laser-cutting window 56, 58 and associated laser beam (not shown).

Two cutting windows and two corresponding laser beams can be obtained using a single laser (e.g. a CO₂ laser) with a beam splitter separating the laser beam between two 2-Axis or 3-Axis scanners, each associated with a corresponding laser-cutting window. In another example, an active beam splitter, e.g. an acousto-optic modulator, can be used to alternately direct a single laser beam to a selected one of two 3-Axis scanners to produce two pulsed beams. In an alternate embodiment, it can be preferred to use two lasers rather than a beam splitter, the two lasers each being associated to a corresponding scanner and cutting window for example. The scanning heads can communicate with an encoder which measures the travelling speed of the web and with a memory which contains information as to the shape to be cut and distance between adjacent shapes. Using a beam splitter in such a system can be compared to using two independent lasers, as will be understood by those skilled in the art.

A laser-cutting process such as described and illustrated herein can be useful in cutting many different materials provided in the form of a web. To name one example, such a laser-cutting process can be used in a production line for personal hygiene products, such as diapers for instance, in which a system having a beam splitter and two 200 W heads programmed for a marking speed of about 5 m/s was found to provide satisfactory cut quality, for instance.

As will be understood, the examples described above and illustrated are intended to be exemplary only. The scope is indicated by the appended claims.

What is claimed is:

1. A process of laser-cutting a shape in a moving web, the process comprising:
moving the web across a laser-cutting window in a longitudinal direction while maintaining a main tension in the web, the main tension being in the longitudinal direction;
applying a specific tension to at least a portion of the web; and
cutting a portion of the shape by moving the laser beam along the portion of the shape, within the laser-cutting window, during said moving of the web, and while maintaining at least a portion of the specific tension at the laser beam in an orientation different than the instantaneous orientation of movement of the laser beam.

2. The process of claim 1 wherein the portion of the shape is a subsequent portion of the shape, further comprising, prior to the step of cutting a subsequent portion of the shape, cutting a former portion of the shape by moving the laser beam along the former portion of the shape, within the laser-cutting window, during said moving of the web, and thereby freeing a portion of the web from the main tension, wherein the step of applying a specific tension includes pulling said freed portion of the web from the rest of the web.

3. The process of claim 2 wherein the subsequent portion of the shape is at least partially longitudinally aligned with the former portion of the shape.

4. The process of claim 1, wherein the step of applying a specific tension includes applying the specific tension in an orientation different than the longitudinal orientation, and wherein the step of cutting a portion of the shape includes moving the laser beam in the longitudinal orientation.

5. The process of claim 1, wherein said cutting further comprises separating the portion of the shape from the web with the specific tension.

6. The process of claim 2, wherein said applying a specific tension to said freed portion of the web further comprises separating the freed portion from the web with the specific tension.

7. The process of claim 1, wherein said applying a specific tension to at least a portion of the web further comprises applying a specific tension having a component in a plane of the web transversally oriented therefrom.

8. The process of claim 1, wherein maintaining at least a portion of the specific tension at the laser beam in an orientation different than the instantaneous orientation of movement of the laser beam further comprises maintaining the specific tension at an acute angle with the instantaneous orientation of the movement of the laser beam.

9. The process of claim 1, wherein said applying a specific tension to at least a portion of the web further comprises applying a mirrored specific tension to the web, the mirrored specific tension having a transversal component compensating for a transversal component of the specific tension.

10. The process of claim 2, wherein said cutting a former portion of the shape by moving the laser beam along the former portion of the shape further comprises allowing a satisfactory amount of the specific tension to be applied on the freed portion of the shape of the web prior to the step of cutting a subsequent portion of the shape.

11. The process of claim 2 wherein said step of applying a specific tension to said freed portion of the web includes engaging said freed portion of the web between nip rollers.

12. A system of laser-cutting a shape in a moving web, the system comprising:
means for moving the web across a laser-cutting window in a longitudinal direction while maintaining a main tension in the web, the main tension being in the longitudinal direction;
means for applying a specific tension to at least a portion of the web; and
means for cutting a portion of the shape by moving the laser beam along the portion of the shape, within the laser-cutting window, during said moving of the web, and while said means for applying a specific tension maintain at least a portion of the specific tension at the laser beam in an orientation different than the instantaneous orientation of movement of the laser beam.

13. The system of claim 12 wherein the portion of the shape is a subsequent portion of the shape, further comprising, means for cutting a former portion of the shape, prior to the step of cutting a subsequent portion of the shape, by moving the laser beam along the former portion of the shape, within the laser-cutting window, during said moving of the web, and thereby freeing a portion of the web from the main tension, wherein the means for applying a specific tension can pull said freed portion of the web from the rest of the web.

14. The system of claim 13 wherein the subsequent portion of the shape is at least partially longitudinally aligned with the former portion of the shape.

15. The system of claim 12, wherein the means for applying a specific tension include means for applying the specific tension in an orientation different than the longitudinal orientation, and wherein the means for cutting a portion of the shape include means for moving the laser beam in the longitudinal orientation.

16. The system of claim 12, wherein said means for cutting further comprises means for separating the portion of the shape from the web with the specific tension.

17. The system of claim 13, wherein said means for applying a specific tension to said freed portion of the web further comprises means for separating the freed portion from the web with the specific tension.

18. The system of claim 12, wherein said means for applying a specific tension to at least a portion of the web further comprises means for applying a specific tension having a component in a plane of the web transversally oriented therefrom.

19. The system of claim 12, wherein said means for applying a specific tension to at least a portion of the web further comprises means for applying a specific tension to at least a portion of the web at an acute angle with the instantaneous orientation of the movement of the laser beam.

20. The system of claim 12, wherein said means for applying a specific tension to at least a portion of the web further comprises means for applying a mirrored specific tension to the web, the mirrored specific tension having a transversal component compensating for a transversal component of the specific tension.

21. The system of claim 12 wherein said means for applying a specific tension to said freed portion of the web includes nip rollers receiving said freed portion of the web therebetween.
